(19) United States
(12) Patent Application Publication
(10) Pub. No.: US 2023/0134492 A1
(43) Pub. Date: May 4, 2023

(54) INFORMATION PROCESSING APPARATUS, INFORMATION PROCESSING METHOD, PROGRAM, AND INFORMATION PROCESSING SYSTEM

(71) Applicant: SONY GROUP CORPORATION, TOKYO (JP)

(72) Inventors: TOMOYUKI OOTSUKI, TOKYO (JP); JUNICHIRO ENOKI, TOKYO (JP); YOSHIO SOMA, TOKYO (JP); MOTOAKI KOBAYASHI, TOKYO (JP)

(21) Appl. No.: 17/906,407

(22) PCT Filed: Mar. 16, 2021
(86) PCT No.: PCT/JP2021/010604

§ 371 Date: Sep. 15, 2022

(30) Foreign Application Priority Data
Mar. 30, 2020 (JP) 2020-061542

(51) Int. Cl. A61B 3/135; G16H 10/40

(52) U.S. Cl. CPC A61B 3/135; G16H 10/40

(57) ABSTRACT

An information processing apparatus according to an embodiment of the present technology includes an assessment unit and a setting unit. The assessment unit performs weighting on an examination item relating to an examination with an ophthalmic microscope on the basis of medical information relating to a patient. The setting unit sets an operation relating to the examination with the ophthalmic microscope on the basis of the weighting of the examination item. Accordingly, the usability of the ophthalmic microscope can be improved.

### TECHNICAL FIELD

The present technology relates to an information processing apparatus, an information processing method, a program, and an information processing system that can be applied to a slit lamp microscope.

### BACKGROUND ART

In a slit lamp microscope described in Patent Literature 1, setting values of an illumination optical system and an observation optical system, which correspond to each examination item, are recorded. Setting values for the examination item are executed by selecting an examination item. Accordingly, a slit lamp microscope easy to operate is provided (paragraphs [0029] to [0039], FIG. 1, and the like of Patent Literature 1).

In a slit lamp microscope described in Patent Literature 2, an operation mode associated with a particular disease in advance is specified. An observation imaging system and an illumination system are automatically driven on the specified operation mode. Accordingly, the slit lamp microscope is effectively utilized (paragraphs [0086] and [0092], FIG. 4, and the like of Patent Literature 2).

### CITATION LIST

### Patent Literature

Patent Literature 1: Japanese Patent Application Laid-open No. 2004-194689

Patent Literature 2: Japanese Patent Application Laid-open No. 2019-154826

### DISCLOSURE OF INVENTION

### Technical Problem

In an ophthalmic microscope such as a slit lamp microscope, since there are many items that should be set for an examination and there are also a wide variety of examinations, it is often cumbersome to conduct examinations. Therefore, it is desirable to provide a technology capable of improving the usability of the ophthalmic microscope.

In view of the above-mentioned circumstances, it is an objective of the present technology to provide an information processing apparatus, an information processing method, a program, and an information processing system that are capable of improving the usability of a slit lamp microscope.

### Solution to Problem

In order to accomplish the above-mentioned objective, an information processing apparatus according to an embodiment of the present technology includes an assessment unit and a setting unit.

The assessment unit performs weighting on an examination item relating to an examination with an ophthalmic microscope on the basis of medical information relating to a patient.

The setting unit sets an operation relating to the examination with the ophthalmic microscope on the basis of the weighting of the examination item.

In this information processing apparatus, the weighting is performed on the examination item relating to the examination with the ophthalmic microscope on the basis of medical information relating to the patient. On the basis of the weighting of the examination item, the operation relating to the examination with the ophthalmic microscope is set. Accordingly, the usability of the ophthalmic microscope can be improved.

An information processing method according to an embodiment of the present technology is an information processing method that is executed by a computer system and includes performing weighting on an examination item relating to an examination with an ophthalmic microscope on the basis of medical information relating to a patient.

An operation relating to the examination with the ophthalmic microscope is set on the basis of the weighting of the examination item.

A program according to an embodiment of the present technology causes a computer system to execute the following steps.

A step of performing weighting on an examination item relating to an examination with an ophthalmic microscope on the basis of medical information relating to a patient.

A step of setting an operation relating to the examination with the ophthalmic microscope on the basis of the weighting of the examination item.

An information processing system according to an embodiment of the present technology includes a ophthalmic microscope and an information processing apparatus.

The ophthalmic microscope includes an illumination optical system and an imaging optical system.

The information processing apparatus includes an assessment unit and a setting unit.

The assessment unit performs weighting on an examination item relating to an examination with an ophthalmic microscope on the basis of medical information relating to a patient.

The setting unit sets an operation relating to the examination with the ophthalmic microscope on the basis of the weighting of the examination item.

### BRIEF DESCRIPTION OF DRAWINGS

FIG. 1 A diagram schematically showing the overview of a medical system.

FIG. 2 A block diagram showing a configuration example of the medical system.

FIG. 3 A schematic diagram showing examples of examination items.

FIG. 4 A flowchart showing a control example of weighting.

FIG. 5 A schematic diagram showing an example of examination item GUI.

FIG. 6 A flowchart showing an example of automatic imaging control.

FIG. 7 A flowchart showing an example of weighting by a learning algorithm.

FIG. 8 A block diagram showing a hardware configuration example of an information processing apparatus.

### MODE(S) FOR CARRYING OUT THE INVENTION

Hereinafter, embodiments according to the present technology will be described with reference to the drawings.

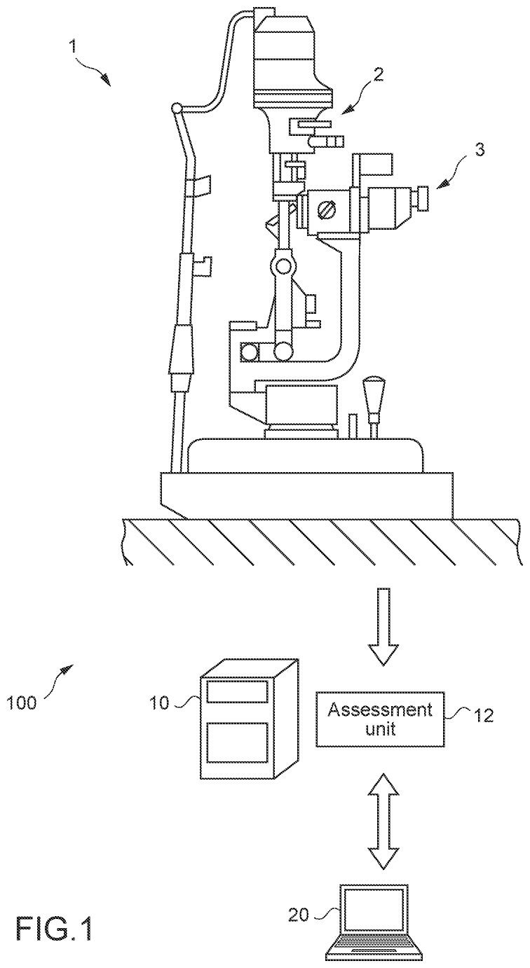

FIG. 1 is a diagram schematically showing the overview of a medical system according to the present technology.

As shown in FIG. 1, a medical system 100 includes a slit lamp microscope 1, an information processing apparatus 10, and a user terminal 20.

Further, in this embodiment, the slit lamp microscope 1 will be exemplified as an ophthalmic microscope. Additionally, various modality devices may be employed as the ophthalmic microscope.

The slit lamp microscope 1 is an apparatus that observes eyes to be examined of a patient. The slit lamp microscope 1 includes an illumination optical system 2 that emits slit light toward an eye to be examined and an imaging optical system 3 that images light reflected by the eye to be examined.

A user (e.g., a doctor) can operate the illumination optical system 2 and the imaging optical system 3 to thereby perform a diagnosis of the patient. For example, the user can control the amount of light of a light source included in the illumination optical system 2, the shape of slit light emitted from the illumination optical system 2, the wavelength of the slit light, the position of the illumination optical system 2, the illumination direction, and the like. For example, the user can also control an observation scale when observing the eye to be examined, the position of the imaging optical system 3, and the like.

As a matter of course, the present technology is not limited thereto, and it may be possible to control a mechanism that controls the position of the patient (eyes to be examined), an imaging timing of the imaging apparatus (e.g., a camera) included in the imaging optical system 3, and the like.

In this embodiment, the shape of the slit light includes any shape. For example, in a case where the shape of the illumination light emitted from the light source (shape of the cross-section orthogonal to the direction of emission to the eye to be examined) is a rectangle, the width and height of the rectangle may be controlled. Moreover, in a case where the shape of the illumination light emitted from the light source is a circle, the radius of the circle may be controlled or the long and short diameters may be controlled to form an elliptical shape.

Further, in this embodiment, also in a case where diffuse light is emitted through an inserted diffusion plate before it is emitted to the eye to be examined, the shape of the diffuse light includes the shape of the slit light.

The information processing apparatus 10 is capable of performing weighting on the examination item relating to an examination with the ophthalmic microscope. In this embodiment, the information processing apparatus 10 performs weighting on the basis of medical information relating to the patient.

The medical information is information for knowing the status of a disease of the patient. In this embodiment, the medical information includes at least one of medical interview information relating to a medical interview to a patient, surgery information relating to a surgical operation performed on the patient, or doctor information relating to a doctor in charge of the patient.

For example, the medical interview information includes the contents of an inquiry answered by the patient, results of the medical interview by the doctor, information regarding the patient's disease and the disease progress that have been input in an electronic medical record or the like, and the like.

Further, for example, the surgical information includes various surgical operations performed on the patient, such as cataract surgery, glaucoma surgery, and vitrectomy. Moreover, the surgical information also includes an examination of a captured image of the eye to be examined of the patient, which has been imaged by another modality device (e.g., a fundus camera), and the like.

Further, for example, the doctor information includes the doctor's name, the order of examinations that the doctor conducts, and the like. It should be noted that in this embodiment, the charge includes a case of conducting medical practice and actions other the medical practice on the patient.

The examination item includes an observation technique using the ophthalmic microscope and an observation condition in the observation technique.

The observation technique is various surgical operations performed on the patient. For example, the observation technique includes any examination such as diffusion, slit lamp biomicroscopy, retro-illumination, funduscopy, gonioscopy, a Van Herick technique, indirect illumination, tonometry, sclerotic scatter, and specular reflection.

The observation condition includes the wavelength, the amount of light, and the illumination direction of the illumination optical system, the observation scale of the imaging optical system, and the like in each observation technique. Additionally, the observation condition may include the width, the height, and the like of slit light.

Further, the information processing apparatus 10 sets an operation relating to the examination with the ophthalmic microscope on the basis of the weighting of the examination item.

The operation is various operations that the ophthalmic microscope can perform. In this embodiment, the operation includes at least one of automatic imaging by the slit lamp microscope 1 or display control of the examination item on an image display unit.

It should be noted that in this embodiment, the automatic imaging includes acquiring a moving image of the eye to be examined in accordance with an examination item selected by the user.

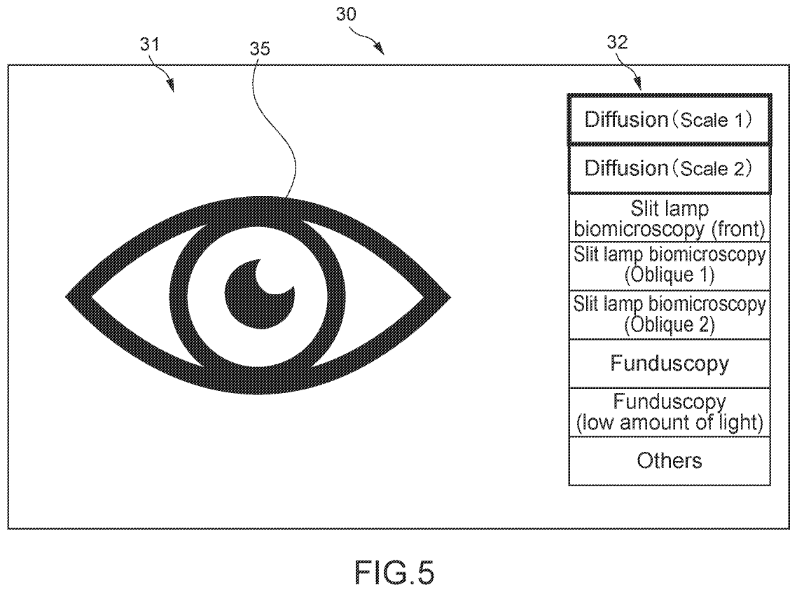

Further, the display control includes causing a display control unit of the user terminal 20 to display a graphical user interface (GUI) that enables an examination item to be selected. A specific example of the GUI will be described referring to FIG. 5.

The user terminal 20 includes various apparatuses that the user can use. In this embodiment, the user is able to select an examination item via the GUI output from the information processing apparatus 10. It should be noted that the user terminal is not limited, and a personal computer (PC), a touch panel, a smartphone, a tablet terminal, or the like having both functions of an image display device and an operation device may be used. Moreover, a monitor that is mounted on the slit lamp microscope 1 as the image display unit may be used. That is, the slit lamp microscope 1 may be used as the user terminal 20.

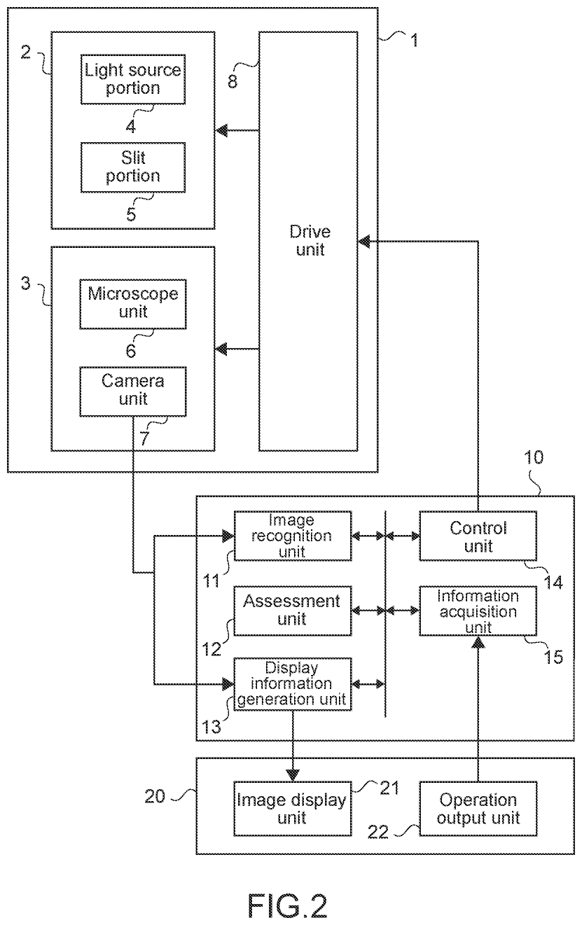

FIG. 2 is a block diagram showing a configuration example of the medical system 100 shown in FIG. 1.

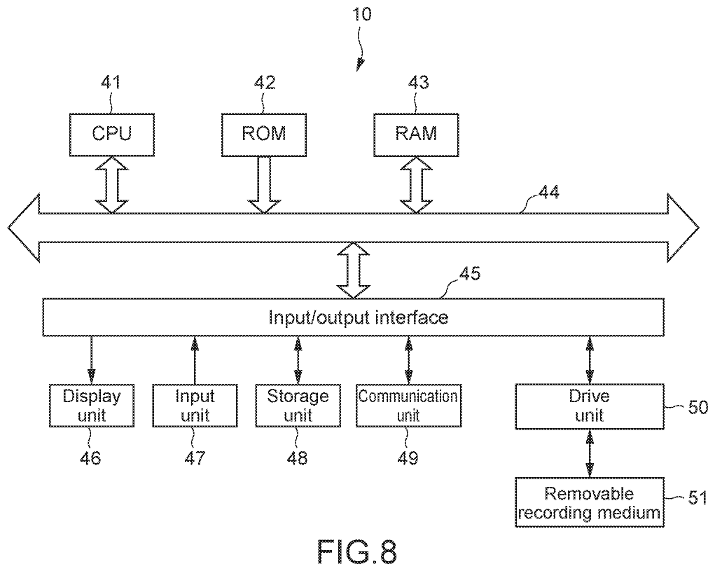

The information processing apparatus 10 includes hardware required for configurations of a computer including, for example, processors such as a CPU, a GPU, and a DSP, memories such as a ROM and a RAM, a storage device such as an HDD (see FIG. 8). For example, the CPU loads a program according to the present technology recorded in the ROM or the like in advance to the RAM and executes the program to thereby execute an information processing method according to the present technology.

For example, any computer such as a PC can realize the information processing apparatus 10. As a matter of course, hardware such as FPGA and ASIC may be used.

In this embodiment, when the CPU executes a predetermined program, an assessment unit as a functional block is configured. As a matter of course, dedicated hardware such as an integrated circuit (IC) may be used for realizing functional blocks.

The program is, for example, installed in the information processing apparatus 10 via various recording media. Alternatively, the program may be installed via the Internet.

The kind of recording medium and the like in which the program is recorded are not limited, and any computer-readable recording medium may be used. For example, any computer-readable non-transitory storage medium may be used.

As shown in FIG. 2, the slit lamp microscope 1 includes the illumination optical system 2, the imaging optical system 3, and a drive unit 8.

The illumination optical system 2 includes a light source portion 4 and a slit portion 5.

The light source portion 4 is a light source of slit light for observing a cornea or fundus of the eye to be examined. For example, the light source portion 4 includes any light source such as a light emitting diode (LED) that outputs constant light and a xenon lamp that outputs flash light. Moreover, for example, a plurality of light sources may be used for the light source portion 4. The present technology is not limited thereto, and the light source portion 4 may include a mechanism that controls the amount of light and the range of illumination.

The slit portion 5 is used for converting illumination light emitted from the light source portion 4 to slit light. For example, the slit portion 5 includes slit blades disposed to be opposite to each other with a predetermined distance. It should be noted that the configuration of the slit portion 5 is not limited, and any configuration may be used. For example, the width of the slit light may be continuously controlled or may be discretely controlled.

Typically, in a case of observing the entire anterior eye part such as an eyelid, a conjunctiva, and a cornea, the slit shape is a circular shape having a large radius. On the other hand, in a case of performing observation of a lens of eye such as a cataract diagnosis, the illumination direction is set to be oblique and a thin slit shape is used for observing slices of the lens of eye. Moreover, in a case of performing funduscopy, the illumination direction is set to be in the front of the eye to be examined for guiding light to the fundus from the pupil and the slit shape is set to have a smaller area for reducing glare to the patient.

The imaging optical system 3 includes a microscope unit 6 and a camera unit 7.

The microscope unit 6 has a configuration for magnifying and observing the eye to be examined. For example, a lens and the like for magnifying the eye to be examined at various scales are used. The configuration of the microscope unit 6 is not limited, and for example, may include eyepieces, a beam splitter, and the like.

The camera unit 7 includes a camera for the right eye and a camera for the left eye that are capable of imaging eyes to be examined. In this embodiment, the camera unit 7 outputs an acquired captured image including an eye to be examined to an image recognition unit 11. It should be noted that the camera unit 7 may include a camera control unit (CCU) or the like that performs image processing on the captured image to be output.

The drive unit 8 controls the illumination optical system 2 and the imaging optical system 3. In this embodiment, the drive unit 8 performs driving control for automatic imaging on the basis of an instruction of automatic imaging from a control unit 14. For example, the drive unit 14 controls the illumination direction of the illumination optical system 2, the imaging timing of the imaging optical system 3, and the like in accordance with the examination item selected via the GUI.

The information processing apparatus 10 includes the image recognition unit 11, an assessment unit 12, a display information generation unit 13, the control unit 14, and an information acquisition unit 15.

The image recognition unit 11 performs image recognition on the basis of the captured image acquired by the camera unit 7. In this embodiment, the image recognition unit 11 is capable of recognizing a disease of the eye to be examined in the captured image. The image recognition unit 11 recognizes a disease of the eye to be examined such as cataract, the position of the disease, and the level of the disease, for example.

Further, in this embodiment, the image recognition unit 11 outputs a recognition result to the assessment unit 12.

The assessment unit 12 assesses the examination item. In this embodiment, the assessment unit 12 performs weighting on the examination item on the basis of the medical information. For example, the assessment unit 12 performs weighting on examination items conducted on the patient in the past.

It should be noted that an assessment method is not limited. For example, assessment points may be added to each piece of medical information by using a rule base, and the weighting may be performed on the basis of a total value. Alternatively, for example, the weighting may be performed using a learning algorithm.

For example, any machine learning algorithm using a deep neural network (DNN) or the like may be used. For example, the accuracy of the weighting of the examination item can be improved by using artificial intelligence (AI) or the like that performs deep learning.

For example, a learning unit and an identification unit are built for performing the weighting of the examination item. The learning unit performs machine learning on the basis of input information (learning data) and outputs a learning result. Moreover, the identification unit performs identification (judgement, prediction, and the like) of the input information on the basis of the input information and the learning result.

For example, a neural network and deep learning may be used for the learning method in the learning unit. The neural network is a model that mimics neural networks of a human brain. The neural network is constituted by three types of layers of an input layer, an intermediate layer (hidden layer), and an output layer.

The deep learning is a model using neural networks with a multi-layer structure. The deep learning can repeat characteristic learning in each layer and learn complicated patterns hidden in mass data.

The deep learning is, for example, used for the purpose of identifying objects in an image or words in a speech. For example, a convolutional neural network (CNN) or the like used for recognition of an image or moving image is used.

Moreover, a neuro chip/neuromorphic chip in which the concept of the neural network has been incorporated can be used as a hardware structure that realizes such machine learning.

Supervised learning, unsupervised learning, semi-supervised learning, reinforcement learning, inverse reinforcement learning, active learning, transfer learning, and the like exist for problem settings in machine learning.

For example, supervised learning learns feature amounts on the basis of provided labeled learning data (training data). Accordingly, labels of unknown data can be derived.

Moreover, unsupervised learning analyzes a large amount of unlabeled learning data, extracts feature amounts, and performs clustering on the basis of the extracted feature amounts. Accordingly, trend analysis and future prediction can be performed on the basis of a huge amount of unknown data.

Moreover, semi-supervised learning is mixed supervised learning and unsupervised learning. The semi-supervised learning is a method in which feature amounts are learned in supervised learning, and then a large amount of training data is provided in unsupervised learning and learning is repeatedly performed while feature amounts are automatically computed.

Moreover, reinforcement learning handles a problem in that an agent in a certain environment observes a current state and determines an action that the agent should take. The agent selects an action to thereby get a reward from the environment and learns a policy that can maximize the reward through a series of actions. In this manner, learning an optimal solution in a certain environment can reproduce the human judgement ability and can also cause a computer to learn a judgement ability beyond the human judgement ability.

Virtual sensing data can also be generated by machine learning. It is possible to predict other sensing data from certain sensing data and uses it as the input information, for example, generate positional information from input image information.

Moreover, it is also possible to generate other sensing data from a plurality of pieces of sensing data. Moreover, it is also possible to predict necessary information and generate predetermined information from the sensing data.

It should be noted that application of the learning algorithm may be performed with respect to any processing in the present disclosure.

The display information generation unit 13 generates a GUI that enables an examination item to be selected. In this embodiment, the display information generation unit 13 displays examination items subjected to weighting by the assessment unit 12. For example, a GUI that makes an examination item capable of specifically examining the patient's disease most outstanding is generated.

The control unit 14 controls the operation of the slit lamp microscope 1. In this embodiment, the slit lamp microscope 1 is caused to perform automatic imaging on the basis of the examination item selected via the user terminal 20. That is, a control signal for causing the slit lamp microscope 1 to execute the observation technique and the observation condition in the selected examination item is output to the drive unit 14.

The information acquisition unit 15 acquires various types of information. For example, the information acquisition unit 15 acquires medical information such as electronic medical records and captured images of the patient in the past, which have been captured by the modality devices, and the user's operation instructions input via the user terminal 20.

In this embodiment, the medical information acquired by the information acquisition unit 15 is output to the assessment unit 14. Moreover, an operation instruction output by an operation output unit 22 is output to the control unit 14.

The user terminal 20 includes an image display unit 21 and the operation output unit 22.

The image display unit 21 includes any image display device such as a projector and a display. In this embodiment, the GUI generated by the display information generation unit 13 is displayed.

The operation output unit 22 outputs an operation instruction input by the user. In this embodiment, when an examination item of the GUI displayed on the image display unit 21 is selected, the operation output unit 22 outputs an operation instruction to perform automatic imaging of the selected examination item.

It should be noted that in this embodiment, the assessment unit 12 corresponds to an assessment unit that performs weighting on an examination item relating to an examination with an ophthalmic microscope on the basis of medical information relating to a patient.

It should be noted that in this embodiment, the display information generation unit 13 and the control unit 14 function as a setting unit that sets an operation relating to the examination with the ophthalmic microscope on the basis of the weighting of the examination item.

It should be noted that in this embodiment, the control unit 14 corresponds to a control unit that causes the ophthalmic microscope to perform the set automatic imaging.

It should be noted that in this embodiment, the display information generation unit 13 corresponds to an output unit that outputs a graphical user interface (GUI) that enables the examination item subjected to weighting by the assessment unit to be selected to the image display unit.

It should be noted that in this embodiment, the image recognition unit 11 corresponds to a recognition unit that recognizes a disease on the basis of the captured image including the eye to be examined of the patient.

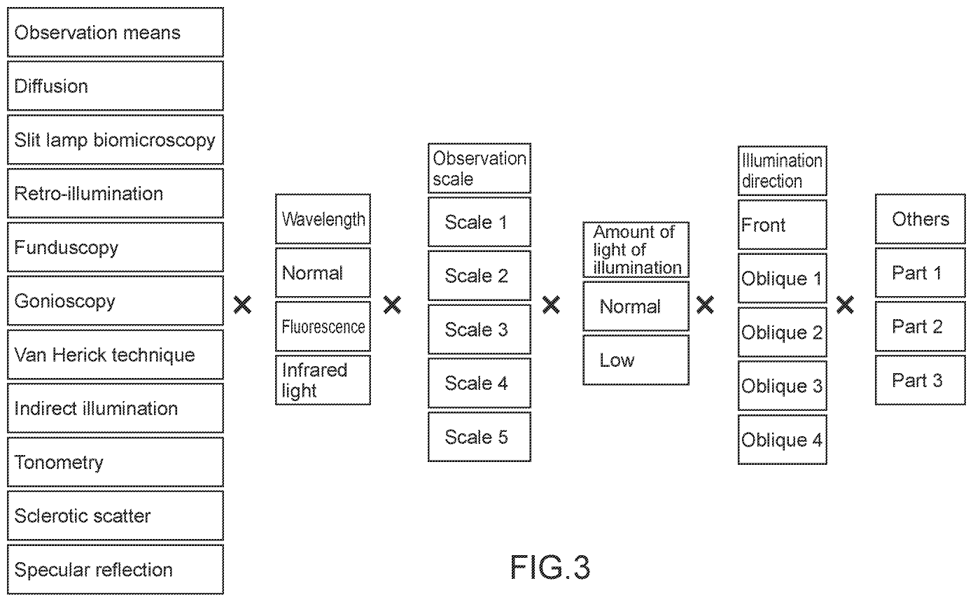

FIG. 3 is a schematic diagram showing examples of the examination items.

As shown in FIG. 3, “diffusion”, “slit lamp biomicroscopy”, “retro-illumination”, “funduscopy”, “gonioscopy”, a “Van Herick technique”, “indirect illumination”, “tonometry”, “sclerotic scatter”, and “specular reflection” are shown as observation techniques.

Further, a “wavelength”, an “observation scale”, an “amount of light of illumination”, an “illumination direction”, and “others” are shown as observation conditions.

In this embodiment, the observation conditions are associated with the observation techniques, respectively. For example, the observation condition that the “wavelength” is normal, the “observation scale” is Scale 2, the “amount of light of illumination” is normal, the “illumination direction” is front, and the “others” are Part 1 is associated with the “diffusion”. Therefore, the number of examination items is at most, for example, the number of items of the observation technique×the number of items of the “wavelength”×the number of items of the “observation scale”×the number of items of the “illumination light”×the number of items of the “illumination direction”×the number of items of the “others”.

In this embodiment, the “wavelength” is a wavelength of the slit light emitted from the illumination optical system 2. For example, for a wavelength pattern of illumination with light emitted from the illumination optical system 2, “normal” where light having a predetermined fixed wavelength is emitted, “fluorescence” where excitation light for a fluorophore such as fluorescein is emitted, and “infrared light” where light having a wavelength corresponding to infrared rays is emitted are set.

The “observation scale” is an observation scale of the imaging optical system 3. For example, settings that “Scale 1” is 1×, “Scale 2” is 2×, “Scale 3” is 4×, “Scale 4” is 8×, “Scale 5” is 16× are made.

The “amount of light of illumination” is an amount of light of the slit light emitted from the illumination optical system 2. For example, “normal” to emit a predetermined fixed amount of light and “low” to emit an amount of light smaller than that of “normal” are each set as the amount of light of illumination.

The “illumination direction” is a direction (angle) slit light emitted toward the eye to be examined. For example, a case of emitting the slit light from a range of from the pupil of the eye to be examined to a predetermined angle is the “front”, a case of emitting the slit light from a state tilted by 15 degrees using the pupil of the eye to be examined as the center is “oblique 1”, a case of emitting the slit light from a state tilted by 30 degrees using the pupil of the eye to be examined as the center is “oblique 2”, a case of emitting the slit light from a state tilted by 45 degrees using the pupil of the eye to be examined as the center is “oblique 3”, and a case of emitting the slit light from a state tilted by 60 degrees using the pupil of the eye to be examined as the center is “oblique 4” as illumination directions.

The “others” are arbitrary settings different from the above-mentioned settings. For example, it may be possible to set the “height” at which the slit light is emitted. Moreover, for example, it may be possible to set the “shape” of the slit light.

It should be noted that the kinds of examination items are not limited, and any observation technique and any observation condition may be set. For example, the imaging time, the aperture of the camera, and the like may be set for the observation technique. Moreover, in a case of an observation technique in which the illumination direction is not set to be larger, such as funduscopy, settings that the illumination direction is large may be excluded.

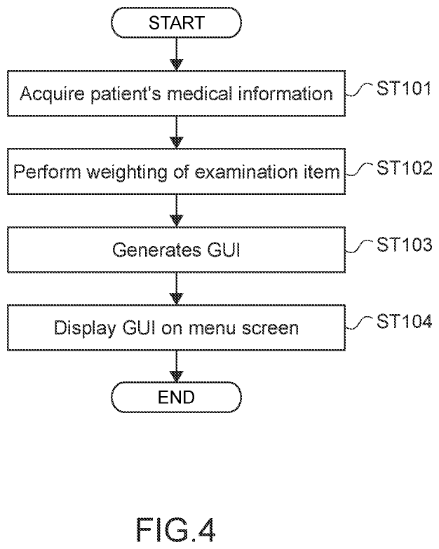

FIG. 4 is a flowchart showing a control example of the weighting.

The information acquisition unit 15 acquires the patient's medical information (Step 101). The assessment unit 12 performs weighting on examination items on the basis of the acquired medical information (Step 102). The display information generation unit 13 generates a GUI that enables an examination item to be selected on the basis of examination items subjected to weighting (Step 103). The generated GUI is displayed on the image display unit 21 of the user terminal 20 (Step 104).

In this embodiment, the examination item to reduce glare to the patient is weighted by the assessment unit 12 in accordance with the patient's disease or the like. For example, the examination item for which the “amount of light of illumination” is set to “low” (observation condition in which the amount of light is set to be smaller than a predetermined threshold), the examination item for which the “wavelength” is set to the “infrared light”, and the like can reduce glare to the patient.

It should be noted that the camera unit 7 may perform, for example, noise reduction by addition averaging where captured images captured with the reduced amount of light are positioned.

FIG. 5 is a schematic diagram showing examples of the examination items GUI.

As shown in FIG. 5, an examination item GUI 30 includes a display unit 31 and an examination item selection unit 32.

The display unit 31 is capable of displaying an eye to be examined 35 to be observed by the slit lamp microscope 1. It should be noted that the eye to be examined 35 to be displayed is not limited, and may be a two-dimensional or three-dimensional captured image or may be a moving image. Moreover, an observation condition or the like associated with an examination item may be displayed on the display unit 31.

The examination item selection unit 32 is capable of displaying the examination items subjected to weighting. In this embodiment, the examination items are displayed in accordance with coefficients of weighting coefficients where weighting has been performed on the basis of the medical information of the patient with respect to the examination items. For example, an examination item having the largest weighting coefficient is displayed in the uppermost row, and examination items are displayed in order in accordance with the weighting coefficients. Typically, examination items that will be conducted for the eye to be examined with a high possibility are displayed.

Further, in this embodiment, the frames are displayed with the thick lines to make the examination items subjected to weighting stand out. Additionally, the frame color may be displayed in a color different from the color of the other examination items or may be displayed with a size larger than the size of the other examination items. That is, the examination item having the largest weighting coefficient may be displayed in the most outstanding state.

Further, in a case where the “others” displayed on the examination item selection unit 32 are selected, examination items not displayed on the examination item GUI 30 are displayed. That is, all examination items are displayed by repeatedly selecting the “others”.

It should be noted that the display configuration on the examination item GUI 30 is not limited. For example, the examination item names may be displayed as short names to be shortly expressed to the user. Moreover, for example, the patient's medical information such as a disease may be displayed or examination items conducted in the past may be displayed.

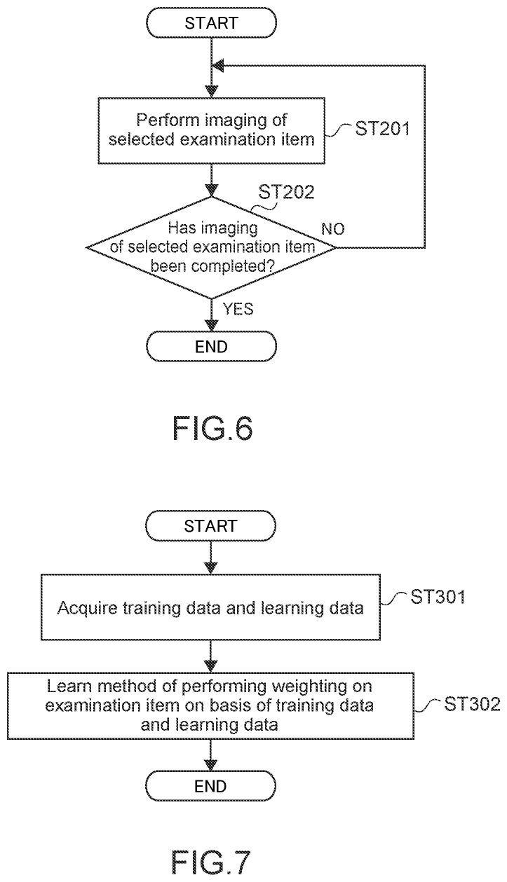

FIG. 6 is a flowchart showing an example of automatic imaging control.

As shown in FIG. 6, the user can cause the slit lamp microscope 1 to perform automatic imaging by selecting an examination item displayed on the examination item GUI 30 (Step 201). Specifically, the control unit 14 outputs to the drive unit 8 a control signal for performing the observation technique and the observation condition associated with the selected examination item. Based on the output control signal, the drive unit 8 controls the illumination optical system 2 and the imaging optical system 3.

Whether or not imaging of the selected examination item has been completed is determined (Step 202), and in a case where the imaging of the selected examination item has been completed (YES in Step 202), the automatic imaging ends. In a case where the user wishes to continue the automatic imaging (NO in Step 202), the automatic imaging is performed by selecting an examination item again.

It should be noted that the assessment unit 12 may perform weighting on the examination item on the basis of the medical information and for example all examination items may be selected so that the weighting becomes equal to or larger than a predetermined threshold, and the control unit 14 may perform automatic imaging for all the selected examination items in sequence on the basis of the results. In this case, in addition, for example, in the assessment unit 12, such an order that examination items having similar observation conditions for the imaging are closer in the imaging order may be determined, information relating to that order may be supplied to the control unit 14, and the control unit 14 may perform automatic imaging in that order. In a case where such automatic imaging of all the examination items is performed, it is not necessarily necessary to present the examination item GUI 30 to the user.

Hereinabove, in the information processing apparatus 10 according to this embodiment, the weighting is performed for examination items relating to the examination with the ophthalmic microscope 1 on the basis of the medical information relating to the patient, and operations relating to the examination with the ophthalmic microscope 1 are set on the basis of the weighting of the examination item. Accordingly, the usability of the ophthalmic microscope can be improved.

Conventionally, in the slit lamp microscope, since there are many items that should be set for an examination and there are also a wide variety of examinations, it is often cumbersome to conduct examinations. In order to solve such a problem, for example, processing in which setting values corresponding to examination items are prestored and setting values are automatically applied by selecting an examination item at the time of an examination is conceivable. However, since there are many examination items, it is cumbersome to select an examination item to be conducted from all the examination items that can be conducted with the slit lamp microscope.

Further, in a case of automatically performing imaging for an examination, since a necessary examination is different for each patient, it is impossible to conduct an examination depending on the patient's disease with the prestored fixed setting values and it is difficult to conduct an efficient examination.

In view of this, in the present technology, the weighting is performed on the examination items on the basis of information for knowing the status of the patient's disease. Moreover, the GUI that enables one of the examination items subjected to the weighting to be selected is displayed, and the automatic imaging is performed by selecting the examination item.

Accordingly, it is less cumbersome to select an examination item when the slit lamp microscope is automatically set up. Moreover, since an examination of a suitable examination item is conducted, it is possible to prevent the user to perform additional imaging because information necessary for the examination is insufficient or the medical examination efficiency from lowering because the examination result includes unnecessary information and the user refers to the unnecessary information. That is, the medical examination efficiency can be improved by acquiring just sufficient results (captured images) when the automatic imaging is performed.

Further, since the examination item to reduce glare is set for the patient in the examination items that can be selected, the burden on a patient sensitive to light due to pigmentary degeneration of the retina or the like can be reduced.

### OTHER EMBODIMENTS

The present technology is not limited to the above-mentioned embodiments, and various other embodiments can be realized.

In the above-mentioned embodiments, the automatic imaging is performed by the user's selection. The present technology is not limited thereto, and the automatic imaging may be performed on the basis of a disease of the eye to be examined, which is acquired in observing the eye to be examined. For example, it is assumed that when the automatic imaging is performed on the eye to be examined in accordance with the examination item of the diffusion (Scale 1) shown in FIG. 5, it is highly probable that the eye to be examined is suffering from cataract according to the image recognition unit 11. In this case, the assessment unit 12 may dynamically add an examination item capable of diagnosing the presence/absence and the level of cataract, such as slit lamp biomicroscopy (front). Moreover, the added examination item may be displayed as the examination item GUI 30 or the automatic imaging may be performed in accordance with the added examination item.

In the above-mentioned embodiments, the weighting of the examination item is performed on the basis of the medical information of the patient. The present technology is not limited thereto, and the weighting may be performed on the basis of a function provided in the slit lamp microscope. For example, in a case of a slit lamp microscope incapable of emitting infrared light, weighting of another examination item excluding the examination item for which the “wavelength” has been set to the “infrared light” is performed.

In the above-mentioned embodiments, the automatic imaging is performed via the examination item GUI 30. The present technology is not limited thereto, and the automatic imaging may be performed by the user uttering words corresponding to the examination item. Moreover, for example, the examination item subjected to weighting may be presented by sound.

In the above-mentioned embodiments, the assessment unit 12 performs weighting on the basis of the medical information of the patient. An algorithm to be used for the weighting at this time is not limited to the fixed one, the weighting may be performed by an algorithm updated as necessary by using a learning algorithm. For example, the information processing apparatus 10 may include a learning unit that generates training data and learning data.

The learning unit uses the patient's medical information as the learning data. Moreover, the learning unit uses images of captured images to which the user has actually referred and the order of referring as the training data. An examination item corresponding to the information of the captured image, which has been stored as the patient's data, may be used as the training data.

It should be noted that information used as the learning data and the training data is not limited. For example, an explicit feed-back result from the user, which relates to the order of conduction examinations or the like may be used as the training data.

FIG. 7 is a flowchart showing an example of the weighting by the learning algorithm.

As shown in FIG. 7, the learning unit acquires the training data and the learning data (Step 101). In this embodiment, the learning unit acquires the patient's medical information (training data) and the order of referring to images (learning data) from the information acquisition unit 15.

The learning unit learns a method of performing weighting on the examination item on the basis of the training data and the learning data. For example, with respect to a patient who is suffering from a predetermined disease, the learning unit performs learning to perform weighting on an examination item selected by a plurality of users many times, which is targeted at the patient, and a learned model is generated.

Further, the contents learned by the learning unit are output to the assessment unit 12. The assessment unit 12 performs weighting on the examination item in accordance with the contents of learning.

It should be noted that the observation condition of the examination item may also be learned. For example, the examination item may be used as the learning data and a corrected observation condition at the time of the doctor's additional examination relating to that examination item, which corresponds to images that the user has referred to or stored may be used as the training data.

It should be noted that learning may be regularly performed or may be performed when it is required by the user.

FIG. 8 is a block diagram showing a hardware configuration example of the information processing apparatus 10.

The information processing apparatus 10 includes a CPU 41, a ROM 42, a RAM 43, an input/output interface 45, and a bus 44 that connects them to one another. A display unit 46, an input unit 47, a storage unit 48, a communication unit 49, and a drive unit 50, and the like are connected to the input/output interface 45.

The display unit 46 is, for example, a display device using liquid-crystal, EL, or the like. The input unit 47 is, for example, a keyboard, a pointing device, a touch panel, or another operation device. In a case where the input unit 77 includes a touch panel, the touch panel can be integral with the display unit 46.

The storage unit 48 is a nonvolatile storage device and is, for example, an HDD, a flash memory, or another solid-state memory. The drive unit 50 is, for example, a device capable of driving a removable recording medium 51 such as an optical recording medium and a magnetic record tape.

The communication unit 49 is a modem, a router, or another communication device for communicating with the other devices, which are connectable to a LAN, WAN or the like. The communication unit 49 may perform wired communication or may perform wireless communication. The communication unit 49 is often used separately from the information processing apparatus 10.

The information processing by the information processing apparatus 10 having the hardware configuration as described above is realized by cooperation of software stored in the storage unit 48, the ROM 42, or the like with hardware resources of the information processing apparatus 10. Specifically, by loading the program that configures the software to the RAM 43, which has been stored in the ROM 42 or the like, and executing the program, the information processing method according to the present technology is realized.

The program is, for example, installed in the information processing apparatus 10 via the recording medium 51. Alternatively, the program may be installed in the information processing apparatus 10 via a global network or the like. Otherwise, any computer-readable non-transitory storage medium may be used.

By cooperation of a computer mounted on a communication terminal with another computer capable of communicating with it via a network or the like, the information processing apparatus, the information processing method, the program, and the information processing system according to the present technology may be executed and the information processing apparatus according to the present technology may be configured.

That is, the information processing apparatus, the information processing method, the program, and the information processing system according to the present technology can be executed not only in a computer system configured by a single computer but also in a computer system in which a plurality of computer operates in cooperation. It should be noted that in the present disclosure, the system means a group of a plurality of components (apparatuses, modules (components), and the like) and it does not matter whether or not all components is in the same casing. Therefore, a plurality of apparatuses housed in separate casings and connected via a network and a single apparatus in which a plurality of modules is housed in a single casing are both systems.

The execution of the information processing apparatus, the information processing method, the program, and the information processing system according to the present technology by the computer system includes, for example, both a case where recording the imaging condition, outputting the GUI, displaying the captured image, and the like are performed by a single computer and a case where the respective processes are performed by different computers. Moreover, execution of the respective processes by a predetermined computer includes causing another computer to performing some or all of the processes to acquire the results.

That is, the information processing apparatus, the information processing method, the program, and the information processing system according to the present technology can also be applied to a cloud computing configuration in which a single function is shared and cooperatively processed by a plurality of apparatuses via a network.

The respective configurations such as the recording unit, the image display unit, and the output unit, the control flows of the communication system, and the like, which have been described with reference to the respective drawings, are merely embodiments, and can be arbitrarily modified without departing from the gist of the present technology. That is, any other configuration, algorithm, and the like for carrying out the present technology may be employed.

It should be noted that the effects described in the present disclosure are merely exemplary and not limitative, and also other effects may be provided. The above descriptions of the plurality of effects do not mean that those effects are always provided at the same time. They mean that at least any one of the above-mentioned effects is provided depending on a condition or the like. As a matter of course, effects not described in the present disclosure can be provided.

At least two feature parts of the feature parts of the above-mentioned embodiments can also be combined. That is, various feature parts described in each of the above-mentioned embodiments may be arbitrarily combined across those embodiments.

In the present disclosure, it is assumed that the concepts that define the shape, the size, the position relationship, the state, and the like such as “center”, “middle”, “uniform”, “equal”, “the same”, “orthogonal”, “parallel”, “symmetric”, “extending”, “axial”, “columnar”, “cylindrical”, “ring-shaped”, and “annular” are concepts including “substantially center”, “substantially middle”, “substantially uniform”, “substantially equal”, “substantially the same”, “substantially orthogonal”, “substantially parallel”, “substantially symmetric”, “substantially extending”, “substantially axial”, “substantially columnar”, “substantially cylindrical”, “substantially ring-shaped”, “substantially annular”, and the like.

For example, states included in a predetermined range (e.g., ±10% range) using “completely center”, “completely middle”, “completely uniform”, “completely equal”, “completely the same”, “completely orthogonal”, “completely parallel”, “completely symmetric”, “completely extending”, “completely axial”, “completely columnar”, “completely cylindrical”, “completely ring-shaped”, “completely annular”, and the like as the basis are also included.

It should be noted that the present technology can also take the following configurations.

(1) An information processing apparatus, including:

an assessment unit that performs weighting on an examination item relating to an examination with an ophthalmic microscope on the basis of medical information relating to a patient; and

a setting unit that sets an operation relating to the examination with the ophthalmic microscope on the basis of the weighting of the examination item.

(2) The information processing apparatus according to (1), in which

the ophthalmic microscope is a slit lamp microscope.

(3) The information processing apparatus according to (1) or (2), in which

the operation includes at least one of automatic imaging with the ophthalmic microscope or display control of the examination item on an image display unit.

(4) The information processing apparatus according to any one of (1) to (3), in which

the assessment unit performs weighting on the examination item on the basis of a disease of the patient.

(5) The information processing apparatus according to any one of (1) to (4), in which

the examination item includes an observation technique using the ophthalmic microscope and an observation condition in the observation technique.

(6) The information processing apparatus according to (5), in which

the observation condition includes at least one of a wavelength of illumination light to be used for an eye to be examined, an amount of light of the illumination light, an illumination direction of the illumination light, or an observation scale.

(7) The information processing apparatus according to any one of (1) to (6), in which

the examination item includes at least one of an examination item for which an amount of light of illumination light is set to be smaller than a predetermined threshold or an examination item for which an infrared ray is emitted as the illumination light.

(8) The information processing apparatus according to any one of (1) to (7), in which

the medical information includes at least one of medical interview information relating to a medical interview to the patient, surgery information relating to a surgical operation performed on the patient, or doctor information relating to a doctor in charge of the patient.

(9) The information processing apparatus according to any one of (1) to (8), in which

the setting unit sets automatic imaging in accordance with the examination item subjected to weighting, the information processing apparatus further including

a control unit that causes the ophthalmic microscope to perform the set automatic imaging.

(10) The information processing apparatus according to any one of (1) to (9), in which

the setting unit sets display control to emphasize the examination item on the basis of a weight of the weighting of the examination item.

(11) The information processing apparatus according to any one of (1) to (10), further including

an output unit that outputs to the image display unit a graphical user interface (GUI) that enables the examination item subjected to the weighting by the assessment unit to be selected.

(12) The information processing apparatus according to any one of (1) to (11), further including

a recognition unit that recognizes the disease on the basis of a captured image including an eye to be examined of the patient.

(13) The information processing apparatus according to any one of (1) to (12), in which

the assessment unit performs weighting on the examination item by using a learning algorithm.

(14) An information processing method, including:

by a computer system

performing weighting on an examination item relating to an examination with an ophthalmic microscope on the basis of medical information relating to a patient; and

setting an operation relating to the examination with the ophthalmic microscope on the basis of the weighting of the examination item.

(15) A program that causes a computer system to execute:

a step of performing weighting on an examination item relating to an examination with an ophthalmic microscope on the basis of medical information relating to a patient; and

a step of setting an operation relating to the examination with the ophthalmic microscope on the basis of the weighting of the examination item.

(16) An information processing system, including:

an ophthalmic microscope that includes an illumination optical system and an imaging optical system; and

an information processing apparatus including
an assessment unit that performs weighting on an examination item relating to an examination with an ophthalmic microscope on the basis of medical information relating to a patient, and
a setting unit that sets an operation relating to the examination with the ophthalmic microscope on the basis of the weighting of the examination item.

### REFERENCE SIGNS LIST

1 slit lamp microscope
2 illumination optical system
3 imaging optical system
10 information processing apparatus
11 image recognition unit
12 assessment unit
13 display information generation unit
14 control unit
21 image display unit
30 examination item GUI
100 medical system

1. An information processing apparatus, comprising:
an assessment unit that performs weighting on an examination item relating to an examination with an ophthalmic microscope on a basis of medical information relating to a patient; and
a setting unit that sets an operation relating to the examination with the ophthalmic microscope on a basis of the weighting of the examination item.

2. The information processing apparatus according to claim 1, wherein
the ophthalmic microscope is a slit lamp microscope.

3. The information processing apparatus according to claim 1, wherein
the operation includes at least one of automatic imaging with the ophthalmic microscope or display control of the examination item on an image display unit.

4. The information processing apparatus according to claim 1, wherein
the assessment unit performs weighting on the examination item on a basis of a disease of the patient.

5. The information processing apparatus according to claim 1, wherein
the examination item includes an observation technique using the ophthalmic microscope and an observation condition in the observation technique.

6. The information processing apparatus according to claim 5, wherein
the observation condition includes at least one of a wavelength of illumination light to be used for an eye to be examined, an amount of light of the illumination light, an illumination direction of the illumination light, or an observation scale.

7. The information processing apparatus according to claim 1, wherein
the examination item includes at least one of an examination item for which an amount of light of illumination light is set to be smaller than a predetermined threshold or an examination item for which an infrared ray is emitted as the illumination light.

8. The information processing apparatus according to claim 1, wherein
the medical information includes at least one of medical interview information relating to a medical interview to the patient, surgery information relating to a surgical operation performed on the patient, or doctor information relating to a doctor in charge of the patient.

9. The information processing apparatus according to claim 1, wherein
the setting unit sets automatic imaging in accordance with the examination item subjected to weighting, the information processing apparatus further comprising
a control unit that causes the ophthalmic microscope to perform the set automatic imaging.

10. The information processing apparatus according to claim 1, wherein
the setting unit sets display control to emphasize the examination item on a basis of a weight of the weighting of the examination item.

11. The information processing apparatus according to claim 1, further comprising
an output unit that outputs to the image display unit a graphical user interface (GUI) that enables the examination item subjected to the weighting by the assessment unit to be selected.

12. The information processing apparatus according to claim 1, further comprising
a recognition unit that recognizes the disease on a basis of a captured image including an eye to be examined of the patient.

13. The information processing apparatus according to claim 1, wherein
the assessment unit performs weighting on the examination item by using a learning algorithm.

14. An information processing method, comprising:
by a computer system
performing weighting on an examination item relating to an examination with an ophthalmic microscope on a basis of medical information relating to a patient; and
setting an operation relating to the examination with the ophthalmic microscope on a basis of the weighting of the examination item.

15. A program that causes a computer system to execute:
a step of performing weighting on an examination item relating to an examination with an ophthalmic microscope on a basis of medical information relating to a patient; and
a step of setting an operation relating to the examination with the ophthalmic microscope on a basis of the weighting of the examination item.

16. An information processing system, comprising:
an ophthalmic microscope that includes an illumination optical system and an imaging optical system; and
an information processing apparatus including
an assessment unit that performs weighting on an examination item relating to an examination with an ophthalmic microscope on a basis of medical information relating to a patient, and
a setting unit that sets an operation relating to the examination with the ophthalmic microscope on a basis of the weighting of the examination item.
